(19) United States
(12) Patent Application Publication
(10) Pub. No.: US 2026/0345231 A1
(43) Pub. Date: Nov. 5, 2026

(54) EMBEDDED ELECTRODES AND PARTIAL ELECTRODES

(71) Applicant: Boston Scientific Scimed, Inc., Maple Grove, MN (US)

(72) Inventors: Mirian Kara Lutes, Etobicoke (CA); Brian Ma, Etobicoke (CA); Suresh Vijay Subramaniam, Cambridge (CA); Stephen Peterkin, Mississauga (CA); Luke Patrick Keaveney, Georgetown (CA); Mahban Samiee-Zafarghandy, Oakville (CA); Miles Montgomery, East Gwillimbury (CA); Ginette Grace Hartell, Toronto (CA); Jastyn Balasubramaniam, Brampton (CA)

(21) Appl. No.: 19/668,506

(22) Filed: May 5, 2026

Related U.S. Application Data
(60) Provisional application No. 63/800,182, filed on May 5, 2025.

(51) Int. Cl. A61B 18/14; A61B 18/08; A61B 18/00

(52) U.S. Cl. CPC A61B 18/1492; A61B 18/082; A61B 2018/00178; A61B 2018/00577

(57) ABSTRACT

An elongated medical device includes a liner defining a lumen. An inner polymeric sleeve surrounds the liner. At least one electrode is located on the inner polymeric sleeve. At least one conductor is electrically connected with the at least one electrode. The at least one conductor is configured to transfer electrical signals between the at least one electrode and a control system. An outer polymeric sleeve surrounds the at least one electrode and the inner polymeric sleeve. At least one aperture is formed in the outer polymeric sleeve to expose a portion of the at least one electrode.

### CROSS REFERENCE TO RELATED APPLICATIONS

This application claims priority to U.S. Provisional Patent Application No. 63/800,182 entitled “EMBEDDED ELECTRODES AND PARTIAL ELECTRODES,” filed May 5, 2025, which is hereby incorporated by reference in its entirety.

### TECHNICAL FIELD

The present disclosure relates to methods and systems incorporating electrodes for use in medical procedures. More specifically, the present disclosure relates to methods and systems having electrodes embedded in an elongated medical device or electrodes spanning less than a full circumference of an elongated medical device.

### BACKGROUND

Electrosurgical procedures are used to treat many different conditions in patients. Ablation is one procedure that can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through electrodes located on the probe to the surrounding tissue. The radio frequency waves generate heat, which destroys surrounding tissue and cauterizes blood vessels.

Some probes include expanding assemblies, or cages, having splines that carry one or more electrosurgical electrode for delivering energy to a patent. In addition, the probes or an additional device such as a guide sheath may carry one or more additional electrode useable for sensing a parameter within the body, or for aiding in navigation during the procedure. In some situations, the one or more electrosurgical electrode may contact or come close to the one or more additional electrode during the procedure. This may lead to arcing between the two electrodes, creating heat or steam bubbles, potentially leading to serious patient harm. Additionally, a safety mechanism may be triggered, such as an auto-off, which can cause delay of the procedure. The present disclosure aims to reduce the probability of this potentially dangerous electrode interaction by reducing the exposed surface area of electrodes used for sensing and/or navigation, and positioning them in a location less likely to contact an electrode used for delivering energy to the patent.

### SUMMARY

Example 1 is an elongated medical device. The medical device includes an inner sleeve. The inner sleeve has an outer surface extending between an inner sleeve proximal end and an inner sleeve distal end. At least one electrode located on the inner sleeve. At least one conductor is electrically connected with the at least one electrode. The at least one conductor is configured to transfer electrical signals between the at least one electrode and a control system. An outer sleeve surrounds the at least one electrode and the inner sleeve. At least one aperture is formed in the outer sleeve to expose a portion of the at least one electrode.

Example 2 is the medical device of Example 1, wherein the at least one electrode comprises a solid electrode or a coil electrode.

Example 3 is the medical device of any of Examples 1 or 2, wherein the at least one electrode surrounds an entire outer circumference of the inner sleeve.

Example 4 is the medical device of any of Examples 1 to 3, wherein the at least one electrode surrounds only a portion of an outer circumference of the inner sleeve.

Example 5 is the medical device of any of Examples 1 to 4, wherein the at least one electrode includes a plurality of perforations to enhance attachment of the at least one electrode to the during a reflow procedure.

Example 6 is the medical device of any of Examples 1 to 5, wherein the at least one aperture comprises one or more slit, circle, oval, square, rectangle, or polygon.

Example 7 is the medical device of Example 6, wherein the one or more slit extends along a longitudinal axis of the elongated medical device.

Example 8 is the medical device of Example 6, wherein the one or more slit extends partially around a circumference of the elongated medical device.

Example 9 is the medical device of any of Examples 1 to 8, wherein the at least one aperture exposes at least one edge of the at least one electrode.

Example 10 is the medical device of any of Examples 1 to 9, wherein the at least one aperture comprises a plurality of apertures sharing a common longitudinal axis along the elongated medical device.

Example 11 is the medical device of Example 10, wherein the common longitudinal axis is a neutral axis.

Example 12 is the medical device of Example 10, wherein the common longitudinal axis is opposite of a bend direction of the elongated medical device.

Example 13 is the medical device of any of Examples 1 to 9, wherein the at least one aperture comprises a plurality of apertures arranged at different circumferential locations along the elongated medical device.

Example 14 is the medical device of Example 13, wherein the plurality of apertures expose different portions of a single electrode.

Example 15 is the medical device of Example 13, wherein the plurality of apertures expose portions of a plurality of electrodes.

Example 16 is an elongated medical device. The medical device includes a liner defining a lumen. An inner sleeve surrounds the liner. At least one electrode is located on the inner sleeve. At least one conductor is electrically connected with the at least one electrode. The at least one conductor is configured to transfer electrical signals between the at least one electrode and a control system. An outer sleeve surrounds the at least one electrode and the inner sleeve. At least one aperture is formed in the outer sleeve to expose a portion of the at least one electrode.

Example 17 is the medical device of Example 16, wherein the at least one electrode comprises a solid electrode or a coil electrode.

Example 18 is the medical device of Example 16, wherein the at least one electrode surrounds an entire outer circumference of the inner sleeve.

Example 19 is the medical device of Example 16, wherein the at least one electrode surrounds only a portion of an outer circumference of the inner sleeve.

Example 20 is the medical device of Example 16, wherein the at least one electrode includes a plurality of perforations to enhance attachment of the at least one electrode to the during a reflow procedure.

Example 21 is the medical device of Example 16, wherein the at least one aperture comprises one or more slit, circle, oval, square, rectangle, or polygon.

Example 22 is the medical device of Example 21, wherein the one or more slit extends along a longitudinal axis of the elongated medical device.

Example 23 is the medical device of Example 21, wherein the one or more slit extends partially around a circumference of the elongated medical device.

Example 24 is the medical device of Example 16, wherein the at least one aperture exposes at least one edge of the at least one electrode.

Example 25 is the medical device of Example 16, wherein the at least one aperture comprises a plurality of apertures sharing a common longitudinal axis along the elongated medical device.

Example 26 is he medical device of Example 25, wherein the common longitudinal axis is a neutral axis.

Example 27 is the medical device of Example 25, wherein the common longitudinal axis is opposite of a bend direction of the elongated medical device.

Example 28 is the medical device of Example 16, wherein the at least one aperture comprises a plurality of apertures arranged at different circumferential locations along the elongated medical device.

Example 29 is the medical device of Example 28, wherein the plurality of apertures expose different portions of a single electrode.

Example 30 is the medical device of Example 28, wherein the plurality of apertures expose portions of a plurality of electrodes.

Example 31 is an elongated medical device. The medical device includes a liner defining a lumen. At least one guide member is located along an outer surface of the liner. An inner polymeric sleeve surrounds the liner and the at least one guide member. At least one electrode is located on the inner polymeric sleeve. At least one conductor is located within the at least one guide member. The at least one conductor is electrically connected with the at least one electrode. The at least one conductor is configured to transfer electrical signals between the at least one electrode and a control system. An outer polymeric sleeve surrounds the at least one electrode and the inner polymeric sleeve. At least one aperture is formed in the outer polymeric sleeve to expose a portion of the at least one electrode.

Example 32 is the medical device of Example 31, wherein the at least one aperture comprises a plurality of apertures sharing a common longitudinal axis along the elongated medical device.

Example 33 is the medical device of Example 31, wherein the at least one aperture comprises a plurality of apertures arranged at different circumferential locations along the elongated medical device.

Example 34 is an elongated medical device. The medical device includes a liner defining a lumen. At least one guide member is located along an outer surface of the liner. A support layer surrounds the at least one guide member and the liner. An inner polymeric sleeve surrounds the liner, the at least one guide member, and the support layer. At least one electrode is located on the inner polymeric sleeve. At least one conductor is electrically connected with the at least one electrode. The at least one conductor is configured to transfer electrical signals between the at least one electrode and a control system. An outer polymeric sleeve surrounds the at least one electrode and the inner polymeric sleeve. At least one aperture is formed in the outer polymeric sleeve to expose a portion of the at least one electrode.

Example 35 is the medical device of Example 34 further comprising an inner member movable within the lumen, and an expandable assembly having a distally located central hub portion affixed to the inner member and a plurality of splines.

Example 36 is an elongated medical device. The medical device includes an inner sleeve extending between an inner sleeve proximal end and an inner sleeve distal end. An outer sleeve surrounds the inner sleeve. At least one electrode mounted on the outer sleeve. The at least one electrode has an exposed portion spanning less than a full circumference of the outer sleeve. At least one conductor is electrically connected with the at least one electrode. The at least one conductor is configured to transfer electrical signals between the at least one electrode and a control system.

Example 37 is the medical device of Example 36, wherein an entirety of the at least one electrode spans less than half of the circumference of the outer sleeve.

Example 38 is the medical device of Example 36, wherein an entirety of the at least one electrode spans less than a quarter of the circumference of the outer sleeve.

Example 39 is the medical device of any of Examples 36 to 38,wherein the at least one electrode includes a connector for connecting to the at least one conductor.

Example 40 is the medical device of Example 39, wherein the connector comprises a bent portion at an edge of the at least one electrode or a central channel.

Example 41 is the medical device of any of claims 36 to 40, wherein the at least one electrode comprises a quadrilateral having a curve corresponding to a circumference of the outer sleeve.

Example 42 is the medical device of claim 42, wherein the at least one electrode comprises an elevated central portion separating a first lower side and a second lower side.

Example 43 is the medical device of claim 43, further comprising plurality of openings through the first lower side and the second lower side, the plurality of openings being configured to enhance attachment of the at least one electrode during a reflow procedure.

Example 44 is the medical device of any of claims 36 to 38, wherein the at least one electrode is embossed.

Example 45 the medical device of any of claims 36 to 38, wherein the at least one electrode comprises an upper plate and a lower plate.

Example 46 is the medical device of any of claims 36 to 38, wherein the at least one electrode is formed by an exposed portion of the at least one conductor.

Example 47 is the medical device of any of claims 36 to 46, wherein the at least electrode comprises a plurality of electrodes sharing a common longitudinal axis along the elongated medical device.

Example 48 is the medical device of claim 47, wherein the common longitudinal axis is a neutral axis.

Example 49 is the medical device of claim 47, wherein the common longitudinal axis is opposite of a bend direction of the elongated medical device.

Example 50 is the medical device of any of claims 36 to 46, wherein the at least one electrode comprises a plurality of electrodes arranged at different circumferential locations along the elongated medical device.

Example 51 is an elongated medical device including a liner defining a lumen. The liner extends between a liner proximal end and a liner distal end. An inner sleeve surrounds the liner. An outer sleeve surrounds the inner sleeve. At least one electrode mounted on the outer sleeve. The at least one electrode has an exposed portion spanning less than a full circumference of the outer sleeve. At least one conductor is electrically connected with the at least one electrode. The at least one conductor is configured to transfer electrical signals between the at least one electrode and a control system.

Example 52 is the medical device of Example 51, wherein an entirety of the at least one electrode spans less than half of the circumference of the outer sleeve.

Example 53 is the medical device of Example 51, wherein an entirety of the at least one electrode spans less than a quarter of the circumference of the outer sleeve.

Example 54 is the medical device of Example 51, wherein the at least one electrode includes a connector for connecting to the at least one conductor.

Example 55 is the medical device of Example 54, wherein the connector comprises a bent portion at an edge of the at least one electrode or a central channel.

Example 56 is the medical device of Example 51, wherein the at least one electrode comprises a quadrilateral having a curve corresponding to a circumference of the outer sleeve.

Example 57 is the medical device of Example 56, wherein the at least one electrode comprises an elevated central portion separating a first lower side and a second lower side.

Example 58 is the medical device of Example 57, further comprising plurality of openings through the first lower side and the second lower side, the plurality of openings being configured to enhance attachment of the at least one electrode during a reflow procedure.

Example 59 is the medical device of Example 51, wherein the at least one electrode is embossed.

Example 60 is the medical device of Example 51, wherein the at least one electrode comprises an upper plate and a lower plate.

Example 61 is the medical device of Example 51, wherein the at least one electrode is formed by an exposed portion of the at least one conductor.

Example 62 is the medical device of Example 51, wherein the at least electrode comprises a plurality of electrodes sharing a common longitudinal axis along the elongated medical device.

Example 63 is the medical device of Example 62, wherein the common longitudinal axis is a neutral axis.

Example 64 is the medical device of Example 62, wherein the common longitudinal axis is opposite of a bend direction of the elongated medical device.

Example 65 is the medical device of Example 51, wherein the at least one electrode comprises a plurality of electrodes arranged at different circumferential locations along the elongated medical device.

Example 66 is an elongated medical device having a liner defining a lumen. The liner extends between a liner proximal end and a liner distal end. At least one guide member is located along an outer surface of the liner. An inner polymeric sleeve surrounds the liner and the at least one guide member. An outer polymeric sleeve surrounds the inner polymeric sleeve. At least one electrode mounted on the outer polymeric sleeve. The at least one electrode has an exposed portion spanning less than a full circumference of the outer polymeric sleeve. At least one conductor is located within the at least one guide member. The at least one conductor is electrically connected with the at least one electrode and is configured to transfer electrical signals between the at least one electrode and a control system.

Example 67 is the medical device of Example 66, wherein the at least one electrode comprises a plurality of electrodes sharing a common longitudinal axis along the elongated medical device.

Example 68 is the medical device of Example 66, wherein the at least one electrode comprises a plurality of electrodes arranged at different circumferential locations along the elongated medical device.

Example 69 is an elongated medical device having a liner defining a lumen. The liner extends between a liner proximal end and a liner distal end. At least one guide member is located along an outer surface of the liner. A support layer surrounds the at least one guide member and the liner. An inner polymeric sleeve surrounds the liner, the at least one guide member, and the support layer. An outer polymeric sleeve surrounds the inner polymeric sleeve. At least one electrode is partially embedded in the outer polymeric sleeve. The at least one electrode has an exposed portion spanning less than a full circumference of the outer polymeric sleeve. At least one conductor is located within the at least one guide member. The at least one conductor is electrically connected with the at least one electrode and is configured to transfer electrical signals between the at least one electrode and a control system.

Example 70 is the medical device of Example 69 further comprising an inner member movable within the lumen, and an expandable assembly including a distally located central hub portion affixed to the inner member and a plurality of splines.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2A is a side view of a catheter suitable for use in the electrophysiological system of FIG. 1, in which the catheter is in a collapsed configuration.

FIG. 2B is a perspective view of a distal portion of a catheter with an expandable assembly in a stored configuration, in accordance with embodiments of the disclosure.

FIGS. 2C and 2D illustrate perspective views of a distal portion of a catheter with an expandable assembly in various expanded configurations, in accordance with embodiments of the disclosure.

FIG. 3A is a perspective view of a portion of the catheter illustrating an embedded electrode and components forming the catheter, in accordance with embodiments of the disclosure.

FIG. 3B is a perspective view of an electrode, in accordance with embodiments of the disclosure.

FIG. 4 is a longitudinal cross-sectional view of the catheter of FIG. 3A along line 4-4, in accordance with embodiments of the disclosure.

FIG. 5 is a transverse cross-sectional view of the catheter of FIG. 3A along line 5-5, in accordance with embodiments of the disclosure.

FIG. 6A is a perspective view of a portion of the catheter illustrating an embedded electrode and components forming the catheter, in accordance with embodiments of the disclosure.

FIG. 6B is a perspective view of an electrode, in accordance with embodiments of the disclosure.

FIG. 7 is a longitudinal cross-sectional view of the catheter of FIG. 6A along line 7-7, in accordance with embodiments of the disclosure.

FIG. 8 is a side view of the catheter illustrating an arrangement of apertures exposing portions of embedded electrodes, in accordance with embodiments of the disclosure.

FIG. 9 is a side view of the catheter illustrating an arrangement of apertures exposing portions of embedded electrodes, in accordance with embodiments of the disclosure.

FIG. 10 is a perspective view of a portion of the catheter illustrating a partial electrode and components forming the catheter, in accordance with embodiments of the disclosure.

FIG. 11 is a perspective view of a partial electrode, in accordance with embodiments of the disclosure.

FIG. 12 is a perspective view of a partial electrode, in accordance with embodiments of the disclosure.

FIG. 13A is a perspective view of a partial electrode, in accordance with embodiments of the disclosure.

FIG. 13B is a side view of the partial electrode of FIG. 13A, in accordance with embodiments of the disclosure.

FIG. 14 is a perspective view of a partial electrode, in accordance with embodiments of the disclosure.

FIG. 15 is a perspective view of a partial electrode, in accordance with embodiments of the disclosure.

FIG. 16 is a perspective view of a partial electrode, in accordance with embodiments of the disclosure.

FIG. 17 is a side view of the catheter illustrating an arrangement of partial electrodes, in accordance with embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

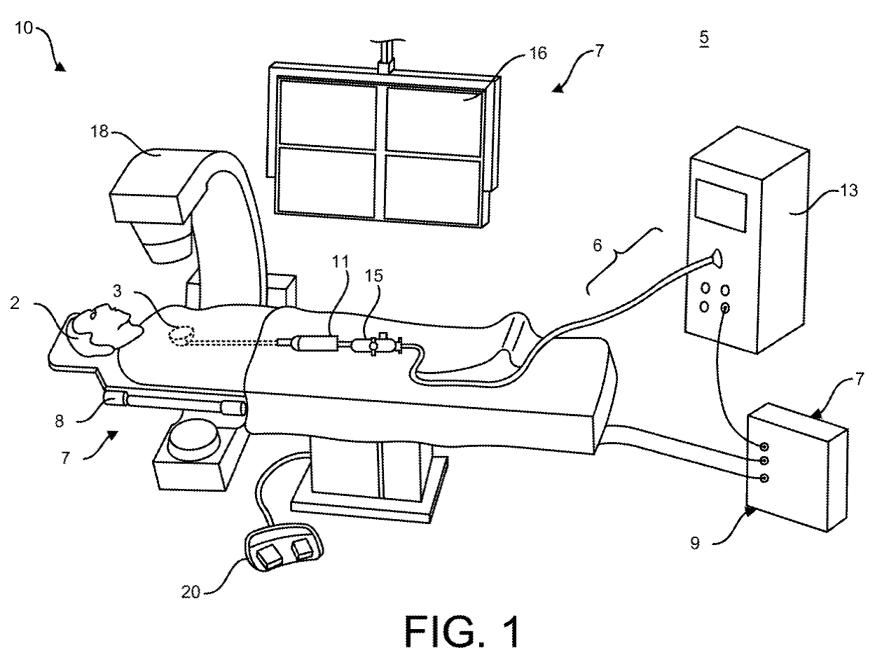

FIG. 1 illustrates an example clinical setting 10 for treating a patient 2, such as for treating a heart 3 of the patient 2, using an electrophysiology system 5, in accordance with the disclosure. The electrophysiology system 5 includes an electroporation catheter system 6 and an electro-anatomical mapping (EAM) system 7. The example electroporation catheter system 6 includes an electroporation catheter 15, an introducer sheath 11, and an electroporation console 13. Additionally, the electroporation catheter system 6 includes various connecting elements, such as cables, that operably connect the components of the electroporation catheter system 6 to one another and to the components of the EAM system 7. In general, the EAM mapping system 7 includes a localization field generator 8, a mapping and navigation controller 9, and a display 16. Also, the clinical setting 10 can include additional equipment such as imaging equipment 18 (represented by the C-arm) and various controller elements, such as a foot controller 20, configured to allow an operator to control various aspects of the electrophysiology system 5. The clinical setting 10 may have other components and arrangements of components that are not shown in FIG. 1. Other arrangements of connecting elements, including wireless connecting elements, are contemplated.

The electroporation catheter system 6 is configured to deliver electric field energy to targeted tissue in the patient’s heart 3 to create cell death in tissue, for example, rendering the tissue incapable of conducting electrical signals. Also, the electroporation catheter system 6 is configured to generate, based on models of electric fields, graphical representations of the electric fields that can be produced using the electroporation catheter 15 and to overlay, on the display 16, the graphical representations of the electric fields or expected or predicted lesions on an anatomical map of the patient’s heart to aid a user in planning ablation by irreversible electroporation using the electroporation catheter 15 prior to delivering energy. In embodiments, the electroporation catheter system 6 is configured to generate the graphical representations of the electric fields based on characteristics of the electroporation catheter 15 and the position of the electroporation catheter 15 in the patient 2, such as in the heart 3 of the patient 2. The electroporation catheter system 6 is configured to generate the graphical representations of the electric fields based on characteristics of the electroporation catheter 15 and the position of the electroporation catheter 15 in the patient 2, such as in the heart 3 of the patient 2, and the characteristics of the tissue surrounding the catheter 15, such as measured impedances of the tissue.

The introducer sheath 11 is operable to provide a delivery conduit through which the electroporation catheter 15 can be deployed to the specific target sites within the patient’s heart 3. Access to the patient’s heart can be obtained through a vessel, such as a peripheral artery or vein. Once access to the vessel is obtained, the electroporation catheter 15 can be navigated to within the patient’s heart, such as within a chamber of the heart.

The example electroporation catheter 15 includes an elongated catheter shaft and distal end configured to be deployed proximate target tissue, such as within a chamber of the patient’s heart. The distal end includes an electrode array to effect treatment. The catheter 15 is capable of being formed into a plurality of configurations. For example, if the distal end region of the catheter is within the patient’s vasculature or is within a sheath as a catheter assembly, such as to travel to the patient to the chamber of the heart, the electrode array is in a collapsed configuration to fit within the sheath. Once the catheter has reached the destination in the chamber of the heart, for example, or the sheath is retracted from the distal region of the catheter 15 (or the shaft catheter is extended past the sheath), and the electrode array is arranged in an expanded configuration for use. In one embodiment, the electrode array can assume other configurations, such as an intermediate configuration between the collapsed and expanded configurations, such as an additional use configuration.

The electrode array includes an electrode assembly comprising a plurality of electrodes. For example, the electrode assembly includes a plurality of spaced-apart electrodes or multiple spaced-apart sets or groups of spaced-apart electrodes. In some examples, an electrode, such as a plurality of spaced-apart electrodes, can be deployed on the catheter shaft in addition to electrodes on the electrode array. In one example, the plurality of electrodes can be formed of a conductive, solid-surface, biocompatible material and are spaced-apart across electrical insulators. Each of the plurality of electrodes is electrically coupled to an associated elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be electrically insulated from one another within an electrically insulating sheath along the catheter shaft, such as with an electrically insulating polymer sheath. The lead conductors can be electrically coupled to plug in the proximal region of the electroporation catheter 15, such as a plug configured to be mechanically and electrically coupled to the electroporation console 13 and the EAM system 7, for example, either directly or via intermediary electrical conductors such as cabling.

The electrode assemblies and associated electrodes are configured for, among other things, sensing cardiac electrical signals, ablation, measuring impedance, sensing tissue contact, localization of the electrode assembly within the patient anatomy such as via the EAM system 7, signal reference, and to determine proximity to target tissue within the anatomy. In some embodiments, the catheter 15 is configured for cardiac mapping, and the electrodes are sensing, or mapping, electrodes configured to be used to collect physiological (electrical) signals to be used to generate electroanatomical maps. An example of a physiological signal that the sensing electrode can acquire includes an intracardiac electrogram (ECG) signal. In some embodiments, the catheter 15 can be a mapping and ablation catheter, and the electrodes can include ablation electrodes, or an ablation electrode assembly, that are configured to deliver ablation electric field energy and sensing electrodes, or a sensing electrode assembly, for mapping purposes. The ablation electrodes in embodiments of an electroporation catheter are configured to receive pulsed electrical signals or waveforms from the console 13 and create pulsed electric fields sufficient to ablate target tissue via irreversible electroporation. The sensing electrodes in the electrode assembly can be electrically coupled to a one or more lead conductors that extends the length of the shaft that are configured to carry an electrical signal received at the sensing electrode. In some examples, an electrode in the electrode assembly can be configured to only perform an ablation or the electrode in the electrode assembly can be configured to only perform mapping. In some examples, an electrode can operate as an ablation electrode in an ablation mode of the electrophysiology system 5 and as a sensing electrode in a sensing or mapping mode of the system 5. Some examples of mapping and ablation catheters are smaller in profile or in the volume of the electrode assembly than catheters that just perform mapping, and clinicians can map a given location within the heart with fewer passes across the chamber with mapping catheters than with mapping and ablation catheters.

In one example, the electroporation console 13 is configured to provide an electrical signal, such as a plurality of concurrent or space-apart-time electrical signals, to the electrically connected electroporation catheter 15 along lead conductors to the spaced-apart electrodes. The spaced-apart electrodes are configured to generate a selected electrical field proximate the target tissue, based on the electrical signals from the electroporation console 13, to effect electroporation.

A selected electrical field can be generated with the electrodes configured as ablation electrodes to effect electroporation. A first ablation electrode, or first group of ablation electrodes, can be selected to be an anode and a different, second ablation electrode, or second group of ablation electrodes, can be selected to be a cathode, such that electrical fields can be generated between the anode and cathode based on signals, such as pulses, provided to the ablation electrodes from the electroporation console 13. The console 13 provides electric pulses of different lengths and magnitudes to the ablation electrodes on the catheter 15. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 13 can select two or more ablation electrodes of the electrode assembly and provides pulses to the selected electrodes to generate electric fields between the selected electrodes to provide pulsed field ablation (PFA). For example, PFA can be performed with monophasic waveforms and biphasic waveforms. Without being bound to a particular theory, electric field strengths in the range of generally 20-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 15 can be operated in a selected mode such as monopolar mode or bipolar mode. During monopolar operation of the catheter 15, an ablation electrode, a group of ablation electrodes, or the entire electrode assembly are configured as one of an anode or a cathode. None of the electrodes in the electrode assembly are configured as the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. During bipolar operation of the catheter 15, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient’s body, but rather through a localized portion of tissue proximate the electrode assembly. For example, the electrodes on the ablation electrode assembly are configured as the one of the anode or cathode and electrodes on the shaft proximate the distal end are configured as the other of the cathode or anode.

The EAM system 7 is operable to track the location of the various components of the electroporation catheter system 6, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 7 can include the RHYTHMIA™ HDx mapping system or the OPAL™ HDx mapping system, both marketed by Boston Scientific Corporation. Also, the mapping and navigation controller 9 of the EAM system 7 includes one or more controllers, such as microprocessors or computers, that execute code out of memory to control or perform functional aspects of the EAM system 7, in which the memory, can be part of the one or more controllers, microprocessors, computers, or part of a memory device accessible through a computer network.

The EAM system 7 generates a localization field, via the field generator 8, to define a localization volume about the heart 3, and a location sensor or sensing element on a tracked device, such as sensors on the electroporation catheter 15, generate an output that can be processed by the mapping and navigation controller 9 to track the location of the sensor, and consequently, the corresponding device, within the localization volume. In the illustrated example, the device tracking is accomplished using magnetic tracking techniques, in which the field generator 8 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 9 to track the location of the various location sensing electrodes within the localization volume.

The EAM system 7 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the RHYTHMIA HDx™ mapping system or the OPAL™ HDx mapping system.

Regardless of the tracking methodology employed, the EAM system 7 utilizes the location information for the various tracked devices, along with cardiac electrical activity acquired by, for example, the electroporation catheter 15 or another catheter or probe equipped with sensing electrodes, to generate, and display via the display 16, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 7 can generate a graphical representation of the various tracked devices within the geometric anatomical map or the electro-anatomical map.

Each cardiac physiological (electrical) signal can include several intracardiac electrograms (EGMs) sensed within a patient's heart and may include any number of features that may be ascertained by aspects of the system 5. Examples of cardiac physiological signal features include activation times, activations, activation waveforms, filtered activation waveforms, minimum voltage values, maximum voltages values, maximum negative time-derivatives of voltages, instantaneous potentials, voltage amplitudes, dominant frequencies, and peak-to-peak voltages. A cardiac physiological signal feature can refer to one or more features extracted from one or more cardiac physiological signals, derived from one or more features that are extracted from one or more cardiac physiological signals. Additionally, a representation, on a cardiac or a surface map, of a cardiac physiological signal feature may represent one or more cardiac physiological signal features, an interpolation of several cardiac physiological signal features. Each cardiac physiological signal also can be associated with a set of respective position coordinates that corresponds to the location at which the cardiac physiological signal was sensed. Each of the respective position coordinates for the sensed cardiac physiological signals can include three-dimensional Cartesian coordinates, polar coordinates, or another coordinate system. The cardiac physiological signals may be sensed on the cardiac surfaces, and the respective position coordinates can be on the endocardial surface, epicardial surface, in the mid-myocardium of the patient's heart, or in a vicinity.

During a signal-acquisition stage of a cardiac mapping procedure, the catheter 15 is displaced to multiple locations within the heart chamber into which the catheter 15 is inserted. At each location to which the catheter 15 is moved, the electrodes and sensors acquire physiological signals resulting from the electrical activity in the heart along with positional, or spatial, information of the catheter 15. External electrodes applied to the patient’s skin create an electric field in the patient’s body. The catheter 15 measures the voltage in different axes within this electric field to determine the position of the catheter electrode within the heart. This positional or spatial information is used in building a three-dimensional grid of the anatomy during mapping. To perform a mapping procedure and reconstruct physiological information on the endocardium surface, the EAM system 7 may align a coordinate system of the catheter 15 with the endocardium surface's coordinate system, or vice versa. Alternatively, or additionally, the grid may be used to capture EGMs and select mapping values based on statistical distributions associated with nodes of the grid. The EAM system 7 also can perform post-processing operations on the physiological information to extract and display useful features of the information to the operator of the system 5.

In generating an example electroanatomical map, a data stream including multiple signals, such as signals received from the mapping electrodes of the catheter 15, is input into the EAM system 7. During the automated electroanatomical mapping process, the data stream provides a collection of physiological and location signals that serve as an input to the mapping process. The signals may be collected directly by the mapping system, obtained from another system using an analog or digital interface, or both. The data stream can include signals such as unipolar and/or bipolar intracardiac EGMs, surface electrocardiograms (ECGs), electrode location information originating from one or more of a variety of methodologies, tissue proximity information, catheter force information, catheter to tissue contact information, catheter temperature, acoustic information, catheter electrical coupling information, catheter deployment shape information, electrode properties, respiration phase, blood pressure, and other physiological information. For the generation of specific types of maps, one or more signals may be used as one or more references to trigger and align the data stream relative to a cycle or clock, which can be used to create beat datasets. Beat metrics can be determined from the beat datasets. A beat acceptance process can be applied to determine which beat datasets will make up a map dataset. The map dataset may be stored in association with a three-dimensional grid that is dynamically generated during data acquisition.

Surface geometry data of the cardiac surface is generated, such as generated concurrently, during the data acquisition process using acceptance metrics employing a surface geometry construction process. This process constructs surface geometry using data such as electrode locations and catheter shape contained in the data stream. Additionally, or alternatively, previously collected surface geometry of the cardiac surface can be used as an input to surface geometry data. Previously collected geometry may have been collected using a different map dataset or using a different modality such as computerized tomography (CT), magnetic resonance imaging (MRI), ultrasound, or rotational angiography and registered to the catheter locating system. A surface map generation process is employed to generate surface map data from the map dataset and surface geometry data.

The depiction of the electrophysiology system 5 shown in FIG. 1 is intended for illustration or a general overview of the various components of the system 5 and is not intended to imply that the disclosure is limited to any set of components or arrangement of the components. For example, additional hardware components, such as breakout boxes or workstations, can be included in the electrophysiology system 5.

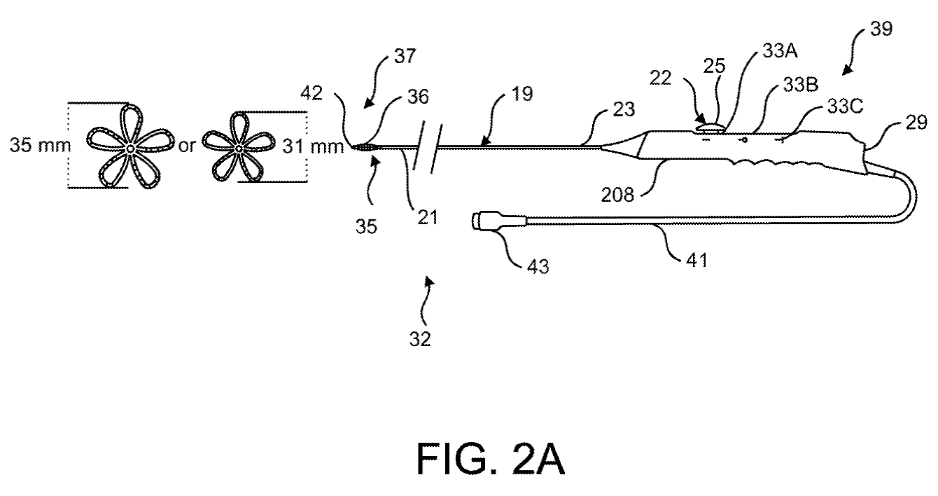

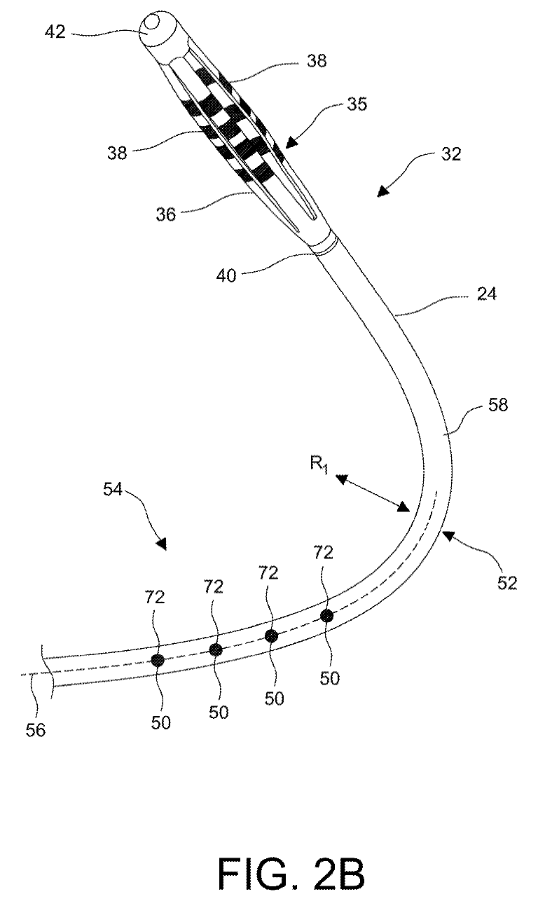

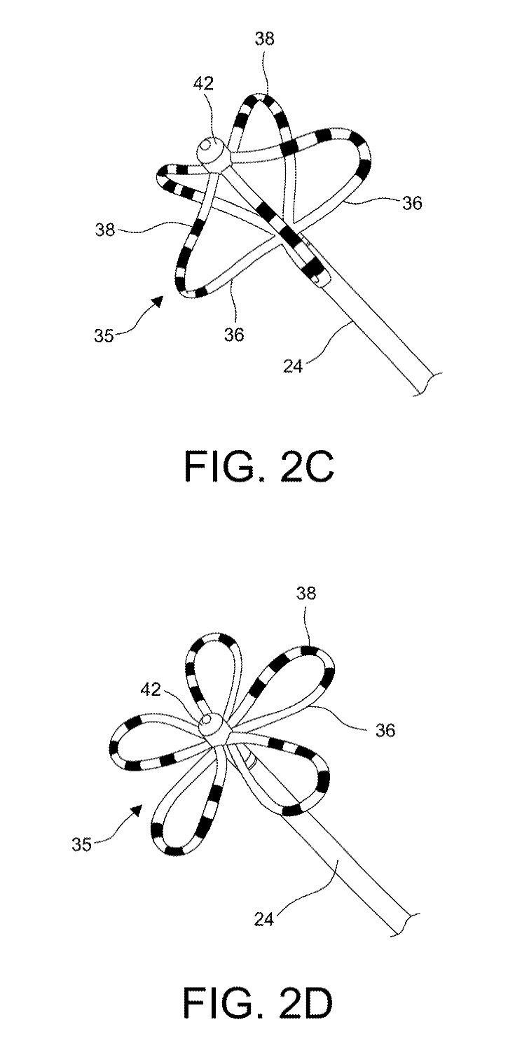

FIG. 2A is a side view of an embodiment of a mapping and ablation catheter 32, which corresponds with catheter 15 of FIG. 1. The catheter 32 can assume a plurality of configurations including a collapsed configuration and an expanded configuration. FIG. 2A illustrates the catheter 32 in the collapsed configuration. The catheter 32 includes an electrode array 35 comprising a set of splines 36 at a distal end 37 of the catheter 32 and a handle 27 at a proximal end 39 of the catheter 32. Each spline of the set of splines 36 includes one or more electrodes 38 formed on a surface of the spline. The electrodes can be configured as ablation electrodes for performing an ablation, sensing electrodes for performing mapping or detection of physiological signals, either ablation or sensing electrodes, or a combination of ablation electrodes and sensing electrodes. Each spline of the set of splines 36 can be configured to include a flexible curvature so as to rotate, or twist and bend and form a petal or tear drop-shaped curve.

The catheter 32 includes a catheter shaft 19 defining a longitudinal axis. The shaft 19 includes a distal region 21 and a proximal region 23. A shaft lumen extends longitudinally through the shaft 19 from the distal region 21 to the proximal region 23 along the longitudinal axis. In some embodiments, a diameter of the catheter shaft 19 is between about 6 French and about 15 French. In certain embodiments, the catheter shaft 19 has a diameter of about 12 French. In some embodiments, the catheter shaft 19 has a length of between about 85 cm and about 135 cm. In certain embodiments, the catheter shaft has a diameter of about 115 cm.

In some examples, a distal cap 42 includes a guidewire lumen, and a guidewire extends along the longitudinal axis in the shaft lumen and through the guidewire lumen in the distal cap 42. The distal cap 42 includes an atraumatic shape in the illustration and can include an electrical component in some embodiments. The set of splines 36 extend from the distal region 21 of the shaft 19. In exemplary embodiments, the set of splines 36 includes a proximal end affixed to the distal region 21 of the shaft 19. The splines include distal ends tethered or fixed to the distal cap 42 such that the set of splines 36 and distal cap 42 can translate relative to the catheter shaft 19 so as to expand and contract together.

In various embodiments, the distal cap 42 translates longitudinally along the guidewire extending through the lumen of the shaft. The set of splines 36 are configured for translation along the longitudinal axis to transition between the collapsed configuration and the expanded configuration. In some examples, the set of splines are configurable in an intermediate configuration for use such as ablation and mapping. The intermediate configuration is between the collapsed configuration and the expanded configuration, and the set of splines can be arranged in, for example, a basket or spherical configuration.

The proximal region 23 of the catheter shaft 19 in some embodiments is coupled to a handle 27 at the proximal end 39 of the catheter 32. The handle 27 in includes a guidewire lumen 29 through the handle 27. The handle 27 is operably coupled to the set of splines 36 and the distal cap 42. For example, the handle 27 includes an actuator 22 disposed in the handle 27. The actuator 22 can include a knob 25, such as a thumb-activated slider, configured to be manipulated translationally or rotationally by a clinician. In the illustrated embodiment, the actuator 22 is configured for translation along the longitudinal axis by moving or sliding the knob 25 between a plurality of positions 33A, 33B, 33C to transition the electrode array 35 between a set of configurations including a collapsed configuration at a collapsed configuration position 33A, the intermediate or basket configuration at an intermediate configuration position 33B, and the expanded configuration at an expanded configuration position 33C. In another embodiment, the actuator 22 can be configured to rotated about the longitudinal axis to transition the catheter 32 between a lock state and an unlock state. The lock state can fix a translational position of the set of splines 36 and the distal cap 42 relative to the catheter shaft 19 and the unlock state can permit translation of the distal cap 42 and set of splines 36 relative to the catheter shaft 19. In some embodiments, the handle 27 includes a flush port (not shown) for saline irrigation. For example, a saline flow may be used to maintain a predetermined level of flow to prevent thrombus formation. The handle 27 can include a cable 41 having connector 43. In some embodiments, the cable 41 is relatively short (e.g., up to about one meter) to increase maneuverability and flexibility of the catheter 32. The connector 43 is configured to couple to an extension cable to connect the catheter 32 to a console, such as console 13, or other components. In some embodiments, connector 43 is electrically connected to electrical components such as the electrodes 38, impedance tracking sensors, a magnetic sensor, temperature sensor, a gyroscopic sensor, an accelerometer via leads disposed in the handle 27, shaft 19, or on the electrode array 35. In some embodiments, the cable 41 and connector 43 can be configured for optical or other signal types.

In some embodiments, the catheter shaft 19 includes an outer member 24 connected to a distal end 26 of the handle 10, and an inner member (not shown) longitudinally moveable within the outer member 24. Movement of the inner member relative to the outer member 24 moves the cap 42 and causes the electrode array 35 to move between a stored and an expanded configuration.

The actuator 22 is configured to be moved by a user’s thumb. As the actuator 22 is moved longitudinally with respect to the housing 12, the inner member moves longitudinally within the outer member 24 to adjust the arrangement of the electrode array 35.

FIG. 2B illustrates an elongated medical device in the form of a catheter 32 for use in an electrosurgical procedure. The catheter 32 includes an electrode array 35, also referred to as an expandable assembly. The electrode array 35 includes a plurality of splines 36 each having at least one electrode 38. The plurality of splines 36 form a basket structure that positions the at least one electrode 38 against a tissue surface within a patent. The embodiment illustrated in FIGS. 2A – 2D includes five splines 36, and each of the five splines 36 includes four electrodes 38. Other embodiments include more or less splines 36 and more or less electrodes 38 to facilitate use in various locations of a patient and to achieve a desired purpose. The at least one electrode 38 can take the form of ablation electrodes, mapping electrodes, pacing electrodes, sensing electrodes, or cauterizing electrodes depending on a procedure to be performed.

The outer member 24 of the catheter 32 includes a distal end 40 that is attached to a proximal end of the plurality of splines 36. A distal end of the plurality of splines 36 is attached to the distal cap 42. In some embodiments, the cap 42 is a separate component. In other embodiments, the cap 42 is formed by molding together the plurality of splines 36. The cap 42 is attached to a distal end of the inner member such that retraction of the inner member causes the plurality of splines 36 to move from a stored configuration as illustrated in FIGS. 2A and 2B to an expanded configuration as illustrated in FIGS. 2C and 2D.

In FIG. 2B, the stored configuration of the electrode array 35 corresponds to the actuator 22 being in a most distal position on the handle 10. In this arrangement, the inner member is also in a most distal position such that the plurality of splines 36 are substantially parallel to a longitudinal axis of the catheter 32.

In FIG. 2C, the electrode array 35 of the catheter 32 is in an expanded configuration where the splines 36 form a bow shape. This arrangement corresponds to the actuator 22 being retracted proximally from the most distal position, which in turn retracts the inner member.

In FIG. 2D, the electrode array 35 of the catheter 32 is in an expanded configuration where the splines 36 form a petal configuration. This arrangement corresponds to the actuator 22 being retracted to a most proximal position on the handle 10, and the inner member it its most proximal position.

The actuator 22 is capable of being moved from a most proximal position to a most distal position, and vice versa, by a user pushing or pulling the actuator. The electrode array 35 of the catheter 32 moves between the stored configuration to the petal configuration as the actuator 22 is translated. A user is able to leave the actuator 22 in any location between the most distal position and the most proximal position in order to expand the splines 36 a desired amount between the stored configuration and the petal configuration.

The catheter 32 is configured to bend along a bending portion 52 that is located proximal of the electrode array 35. The bending portion 52 allows for the electrode array 35 to be directed towards a desired location within a patient, for example an interior of a heart. The bending portion 52 includes a curve having an inner radius R₁ and an outer curve 58. In some embodiments, the inner radius R₁ is fixed. In other embodiments, the inner radius R₁ is adjustable. In some embodiments, the bending portion 52 can include a pre-formed bend having a desired configuration. In other embodiments, the catheter 32 is steerable in response to tension being applied to one or more steering wires (now shown) which extend from near the distal end 40 to the handle 10. In response to tension, the catheter 32 is configured to bend at the bending portion 52 to adjust the shape of the catheter 32. In some embodiments, the catheter 32 is steerable in one direction using a single steering wire. In other embodiments, the catheter 32 is steerable in two opposing directions by two opposing steering wires. In other embodiments, the catheter 32 is steerable in any direction by four uniformly spaced steering wires.

The outer member 24 of the catheter 32 includes at least one electrode 50 located in an intermediate region 54 that is proximal of the bending portion 52. As illustrated in FIG. 2B, the at least one electrode 50 includes four electrodes located along a length of the outer member 24. The at least one electrode 50 is configured as a sensing or navigating electrode. The at least one electrode 50 is electrically connected to a control system, for example an electroanatomical mapping system 7. At least one conductor (not shown in FIG. 2B) is electrically connected with the at least one electrode 50 for transferring electrical signals between the at least one electrode 50 and the control system. The at least one electrode 50 is configured as a partial electrode or an embedded electrode as described below.

In some embodiments, the at least one electrode 50 comprises a plurality of electrodes sharing a common longitudinal axis 56 along the outer member 24. In FIG. 2B, the common longitudinal axis 56 is a neutral axis. A neutral axis is one that resides in a plane that is not in a bending direction of the catheter 32. The neutral axis resides in a plane that is orthogonal to a plane passing through the inner radius R₁ and an outer curve 58. In some embodiments, a plurality of electrodes share a common longitudinal axis that is opposite of a bend direction of the elongated medical device. In FIG. 2B, this axis extends along an outer curve 58 of the bending portion 52.

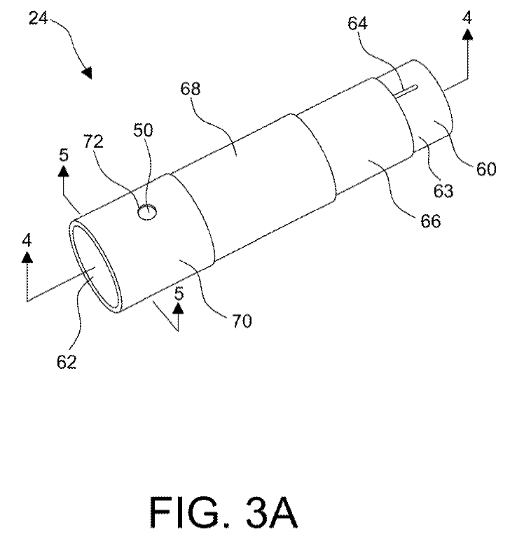

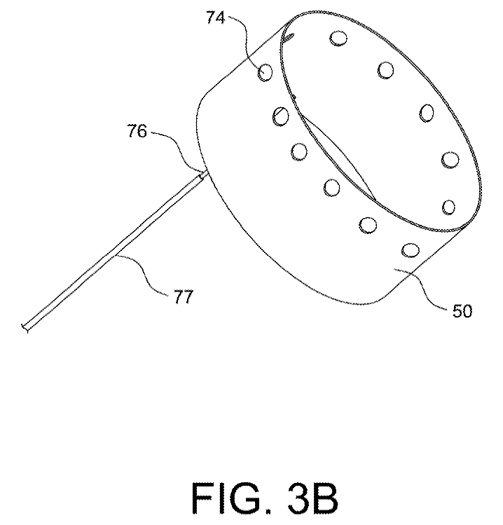

FIG. 3A is a perspective view showing components of a portion of the outer member 24 of the catheter 32 in the intermediate region 54 illustrating at least one electrode 50 as an embedded electrode. While FIG. 3A is directed to the outer member 24 of the catheter 32, it is understood that the outer member 24 can be incorporated into other medical devices or used alone. For example, the outer member 24 can take the form of a dilator, guide sheath, steerable sheath, infusion catheter, therapy catheter, introducer, access device, or any other elongated medical device that incorporates one or more electrode. The outer member 24 is formed by a plurality of layers. The outer member 24 includes a liner 60 defining a lumen 62. The liner 60 is a polymeric liner and is formed of a suitable polymeric material such as Nylon, PEBAX, PTFE, HDPE, UHMWPE, LDPE, and MDPE. At least one guide member 64 is located along an outer surface 63 of the liner 60.

A support layer 66 surrounds the at least one guide member 64 and the liner 60. The support layer 66 includes a braided or coiled structure that provides support to the outer member 24. The support layer 66 is formed of one or more filaments or wires of a suitable polymer, metal, or fabric. Suitable materials include stainless steel, NiTi, Nylon, Kevlar, LCP, PEEK, HDPE. In other embodiments, the support layer 66 can include a hypotube, a slotted hypotube, or a tubular member having at least one molded or laser cut articulating joint. In some embodiments, the support layer 66 entirely surrounds the at least one guide member 64. In other embodiments, the support layer 66 partially surrounds the at least one guide member 64. For example, some filaments forming the support layer 66 may go above the at least one guide member 64 while some filaments forming the support layer 66 go below the at least one guide member 64. In some embodiments, the support layer 66 is positioned entirely beneath the at least one guide member 64. The support layer 66 includes a material that can improve torque transmission, increase rigidity, or otherwise improve handling of the outer member 24. In some embodiments, the support layer 66 includes a consistent braid density or uniform coil pitch. In embodiments, the support layer 66 has a variable braid density or variable coil pitch. In some embodiments, the support layer 66 is formed of a slotted hypotube or tubular structure with a plurality of slots or perforations.

The at least one guide member 64 is an elongated tubular member arranged substantially parallel to a longitudinal axis of the outer member 24. The at least one guide member 64 can have a circular, rectangular, oval, square, or polygonal cross-section. The at least one guide member 64 is formed at least partially of a polymeric material, for example PEBAX, PTFE, polyimide, or a composite thereof. In some embodiments, the at least one guide member 64 includes a reinforcement braid or coil formed of Nylon or polyurethane. In some embodiments, the at least one guide member 64 is configured to guide at least one conductor from the handle 10 to at least one electrode 50. The at least one conductor electrically couples the at least one electrode 50 to a control system, for example an EAM system or an RF generator. In some embodiments, multiple conductors are located in a single guide member 64. In other embodiments, each guide member 64 houses a single conductor.

In other embodiments, the at least one guide member 64 is configured to guide at least one steering wire from the handle 10 to the distal end 40 of the outer member 24. The at least guide member 64 is configured to provide a channel for at least one steering wire that allows for deflection or changing shape of the outer member 24. The at least one steering wire is secured near the distal end 40 and extends proximally through the at least one guide member 64 to the handle 10. A user manipulates a control mechanism (not shown) to cause tensioning of at least one steering wire to deflect or change the shape of the outer member 24.

While not illustrated, the at least one guide member 64 can include a plurality of guide members spaced around the liner 60. In some embodiments, the plurality of guide members are equally spaced around the liner 60. In some embodiments, the plurality of guide members are not spaced equally around the liner 60. In other embodiments, the plurality of guide members are adjacent one another. In some embodiments, the plurality of guide members are arranged in a plurality of groups around the liner 60. In some embodiments, the at least one guide member 64 includes a plurality of guide members, some of which guide a plurality of conductors, and others which guide a plurality of steering wires. Guide members 64 that contain at least one steering wire may be located beneath the support layer 66 while guide members 64 that contain at least one conductor may be located anywhere within the outer member 24. For example, guide members 64 containing at least one conductor may be positioned above the support layer 66, within an inner polymeric sleeve 68, above an inner polymeric sleeve 68, or within an outer polymeric sleeve 70.

An inner polymeric sleeve 68 at least partially surrounds the liner 60, the at least one guide member 64, and the support layer 66. The inner polymeric sleeve 68 electrically insulates the support layer. The inner polymeric sleeve 68 can be applied over the liner 60, the at least one guide member 64, and the support layer 66 using a reflow process to encase the components. The inner polymeric sleeve 68 includes any polymer or polymeric material that can flow in spaces between the support layer 66 and the at least one guide member 64. Some suitable materials for forming at least a portion of the inner polymeric sleeve 68 include Nylon, PEBAX, PTFE, Polyurethane, and PET. In some embodiments, the inner polymeric sleeve 68 covers only a portion of the liner 60, for example at locations corresponding to at least one electrode 50.

At least one electrode 50 is located on the inner polymeric sleeve 68. The at least one electrode 50 is formed of an electrically conductive material. In some embodiments, the at least one electrode is formed of a metal such as a platinum-iridium alloy, gold, other platinum alloys, stainless steel, nickel-titanium or any other biocompatible medical metals. In some embodiments, the at least one electrode is formed of a conductive polymer or a polymer containing conducive particles. In some embodiments, the at least one electrode 50 is a ring electrode that surrounds an entire outer circumference of the inner polymeric sleeve 68. In other embodiments, the at least one electrode 50 surrounds only a portion of an outer circumference of the inner polymeric sleeve 68.

An outer polymeric sleeve 70 surrounds the at least one electrode 50 and the inner polymeric sleeve 68. In some embodiments, the outer polymeric sleeve 70 is formed of a polymer having a lower melting temperature than the polymer forming the inner polymeric sleeve 68. The outer polymeric sleeve 70 provides a smooth outer surface for the outer member 24. In some embodiments, the outer polymeric layer 70 is selected to include one or more materials that increase lubricity of the catheter 32. In some embodiments, a coating (not shown) is applied over the outer polymeric sleeve 70 to increase lubricity of the medical device. In some embodiments, the outer polymeric sleeve 70 and the inner polymeric sleeve 68 are formed of the same polymer or polymeric material. In some embodiments, the outer polymeric sleeve 70 and the inner polymeric sleeve 68 are formed of different polymers or polymeric materials.

At least one aperture 72 is formed in the outer polymeric sleeve 70 over at least one electrode 50 to expose a portion of the at least one electrode 50. The at least one aperture 72 is created mechanically or chemically. For example, the at least one aperture 72 may be formed by griding, shaving, cutting, laser etching, laser ablation, laser drilling, drilling, skiving, and chemical etching among others. The at least one aperture 72 is formed by removing a portion of the outer polymeric sleeve 70 overlaying the at least one electrode 50. As illustrated in FIG. 2B, the at least one aperture 72 includes four apertures exposing portions of four electrodes 50.

FIG. 3B is a perspective view of an electrode 50, in accordance with embodiments of the disclosure. The electrode 50 illustrated in FIG. 3B is a solid ring electrode that is configured to surround an entire outer circumference of the inner polymeric sleeve 68. A conductor 76 is mechanically and electrically coupled to the electrode 50 and extends proximally from the electrode 50 to electrically connect to a control system. The conductor 76 includes a layer of insulation 77. The conductor 76 is coupled to the electrode 50 by an adhesive, welding, crimping, or other method that ensures permanent connection between the electrode 50 and the conductor 76. In some embodiments, the conductor 76 passes through a guide member 64. In other embodiments, the conductor 76 is helically wrapped around the liner 60, helically embedded in the inner polymeric sleeve 68, or helically embedded in the outer polymeric sleeve 70 to create a strain relief to allow the conductor 76 to move as the outer member 24 bends.

The electrode 50 includes a plurality of perforations 74 that extend through the electrode 50. The perforations 74 are configured to enhance attachment of the 50 electrode during a reflow procedure by allowing polymer to flow into the perforations 74. While the perforations 74 are illustrated as round holes, the perforations 74 can include any shape such as square, oval, or slotted for example.

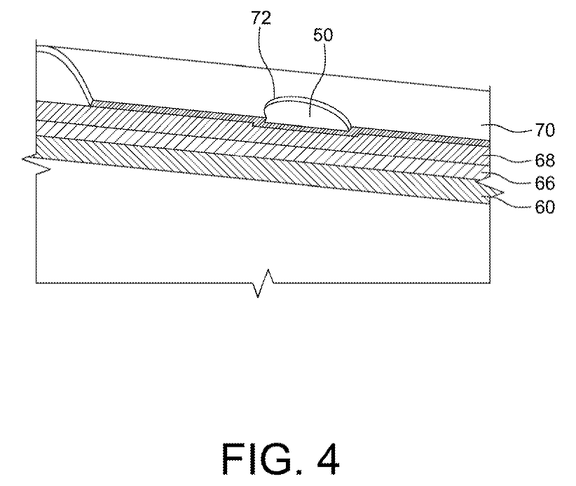

FIG. 4 is a longitudinal cross-sectional view of the catheter 32 of FIG. 3A along line 4-4, in accordance with embodiments of the disclosure. As illustrated in FIG. 4, only a portion of the electrode 50 is exposed by the aperture 72.

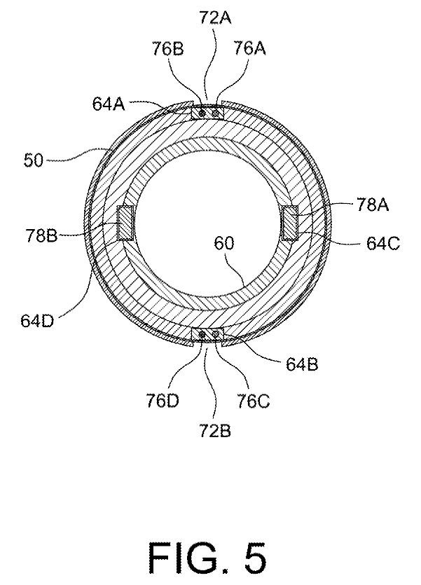

FIG. 5 is a transverse cross-sectional view of the catheter of FIG. 3A along line 5-5, in accordance with embodiments of the disclosure. As illustrated in FIG. 5, the at least one guide member 64 includes four guide members 64A, 64B, 64C, 64D arranged at equal distances around the liner 60. A first guide member 64A and a second guide member 64B located on opposite sides of the liner 60 provide channels for a plurality of conductors 76A, 76B, 76C, 76D. Each of the plurality of conductors 76A, 76B, 76C, 76D are electrically and mechanically coupled to at least one electrode (not shown).

A third guide member 64C and a fourth guide member 64D located on opposite sides of the liner 60 provide channels for a plurality of steering wires 78A, 78B. The first steering wire 78A and the second steering wire 78B are positioned on opposite sides of the line 60 to allow for deflection of the medical device 200 in two directions. While only four guide members 64A, 64B, 64C, 64D are illustrated in FIG. 5, it is understood that increasing the number of guide members would provide more channels for more conductors or more steering wires. Increasing the number of steering wires would allow for an increase in steering capabilities of the catheter 32.

As illustrated in FIG. 5, the electrode 50 is exposed in two diametrically opposed locations by a first aperture 72A and a second aperture 72B. In this embodiment, the first aperture 72A is aligned with the first guide member 64A and the second aperture 72B is aligned with the second guide member 64B. In other embodiments, the electrode 50 is exposed at aperture locations that do not align with any guide member.

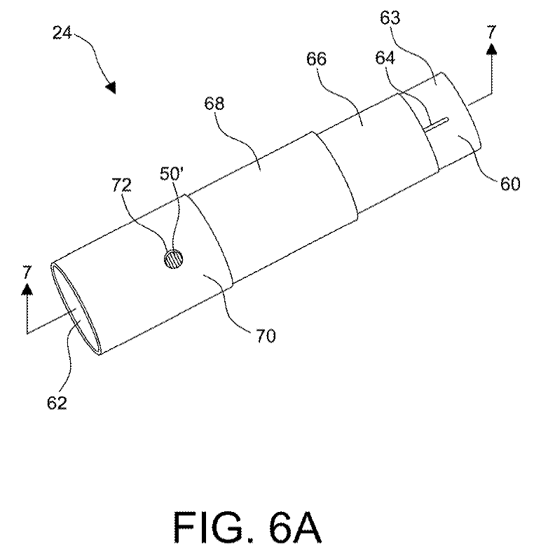

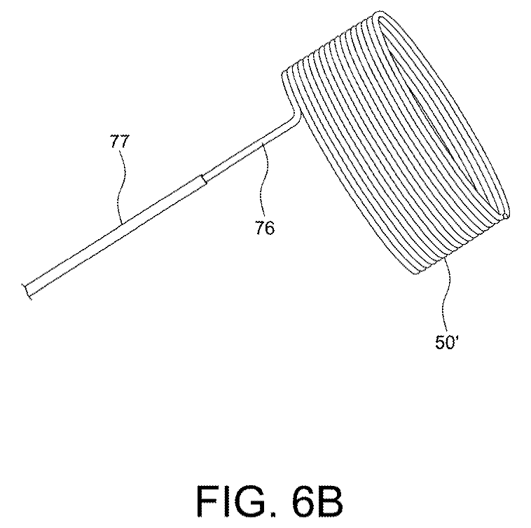

FIG. 6A is a perspective view showing components of a portion of the outer member 24 of the catheter 32 in the intermediate region 54 illustrating at least one electrode 50’ as an embedded electrode. The outer member 24 is formed by a plurality of layers. The outer member 24 includes a liner 60 defining a lumen 62. The liner 60 is a polymeric liner and is formed of a suitable polymeric material such as Nylon, PEBAX, PTFE, HDPE, UHMWPE, LDPE, and MDPE. At least one guide member 64 is located along an outer surface 63 of the liner 60.

A support layer 66 surrounds the at least one guide member 64 and the liner 60. The support layer 66 includes a braided or coiled structure that provides support to the outer member 24. The support layer 66 is formed of one or more filaments or wires of a suitable polymer, metal, or fabric. Suitable materials include stainless steel, NiTi, Nylon, Kevlar, LCP, PEEK, HDPE. In other embodiments, the support layer 66 can include a hypotube, a slotted hypotube, or a tubular member having at least one molded or laser cut articulating joint. In some embodiments, the support layer 66 entirely surrounds the at least one guide member 64. In other embodiments, the support layer 66 partially surrounds the at least one guide member 64. For example, some filaments forming the support layer 66 may go above the at least one guide member 64 while some filaments forming the support layer 66 go below the at least one guide member 64. The support layer 66 includes a material that can improve torque transmission, increase rigidity, or otherwise improve handling of the outer member 24. In some embodiments, the support layer 66 includes a consistent braid density or uniform coil pitch. In embodiments, the support layer 66 has a variable braid density or variable coil pitch. In some embodiments, the support layer 66 is formed of a slotted hypotube or tubular structure with a plurality of slots or perforations.

The at least one guide member 64 is an elongated tubular member arranged substantially parallel to a longitudinal axis of the outer member 24. The at least one guide member 64 can have a circular, rectangular, oval, square, or polygonal cross-section. The at least one guide member 64 is formed at least partially of a polymeric material, for example Pebax, PTFE, or a composite thereof. In some embodiments, the at least one guide member 64 is configured to guide at least one conductor from the handle 10 to at least one electrode 50’. The at least one conductor electrically couples the at least one electrode 50’ to a control system, for example an EAM system or an RF generator.

In other embodiments, the at least one guide member 64 is configured to guide at least one steering wire from the handle 10 to the distal end 40 of the outer member 24. The at least guide member 64 is configured to provide a channel for at least one steering wire that allows for deflection or changing shape of the outer member 24. The at least one steering wire is secured near the distal end 40 and extends proximally through the at least one guide member 64 to the handle 10. A user manipulates a control mechanism (not shown) to cause tensioning of at least one steering wire to deflect or change the shape of the outer member 24.

While not illustrated, the at least one guide member 64 can include a plurality of guide members spaced around the liner 60. In one embodiment, the plurality of guide members are equally spaced around the liner 60. In other embodiments, the plurality of guide members are adjacent to one another. In some embodiments, the at least one guide member 64 includes a plurality of guide members, some of which guide a plurality of conductors, and others which guide a plurality of steering wires.

An inner polymeric sleeve 68 surrounds the liner 60, the at least one guide member 64, and the support layer 66. The inner polymeric sleeve 68 electrically insulates the support layer. The inner polymeric sleeve 68 can be applied over the liner 60, the at least one guide member 64, and the support layer 66 using a reflow process to encase the components. The inner polymeric sleeve 68 includes any polymer or polymeric material that can flow in spaces between the support layer 66 and the at least one guide member 64. Some suitable materials for forming at least a portion of the inner polymeric sleeve 68 include Nylon, PEBAX, PTFE, Polyurethane, and PET.

At least one electrode 50’ is located on the inner polymeric sleeve 68. The at least one electrode 50’ is formed of a coiled wire wrapped to form a ring around the inner polymeric sleeve 68. The wire forming the at least one electrode 50’ is formed of an electrically conductive material. In some embodiments, the wire is formed of a metal such as a platinum-iridium alloy, gold, other platinum alloys, stainless steel, nickel-titanium or any other biocompatible medical metals. In some embodiments, the wire is formed of a conductive polymer or a polymer containing conducive particles.

An outer polymeric sleeve 70 surrounds the at least one electrode 50’ and the inner polymeric sleeve 68. In some embodiments, the outer polymeric sleeve 70 is formed of a polymer having a lower melting temperature than the polymer forming the inner polymeric sleeve 68. The outer polymeric sleeve 70 provides a smooth outer surface for the outer member 24. In some embodiments, the outer polymeric layer 70 is selected to include one or more materials that increase lubricity of the catheter 32. In some embodiments, a coating (not shown) is applied over the outer polymeric sleeve 70 to increase lubricity of the medical device. In some embodiments, the outer polymeric sleeve 70 and the inner polymeric sleeve 68 are formed of the same polymer or polymeric material. In some embodiments, the outer polymeric sleeve 70 and the inner polymeric sleeve 68 are formed of different polymers or polymeric materials.

At least one aperture 72 is formed in the outer polymeric sleeve 70 over at least one electrode 50’ to expose a portion of the at least one electrode 50’. The at least one aperture 72 is created mechanically or chemically. For example, the at least one aperture 72 may be formed by griding, shaving, cutting, laser etching, laser ablation, laser drilling, drilling, skiving, and chemical etching among others. The at least one aperture 72 is formed by removing a portion of the outer polymeric sleeve 70 overlaying the at least one electrode 50’.

FIG. 6B is a perspective view of the electrode 50’ formed of a coil wire, in accordance with embodiments of the disclosure. The electrode 50’ illustrated in FIG. 6B is a ring electrode formed of a coiled wire that surrounds an entire outer circumference of the inner polymeric sleeve 68. The wire forming the electrode 50’ extends proximally from the electrode 50’ to form a conductor 76 to electrically connect to a control system. A layer of insulation 77 surrounds the conductor 76 to prevent electrical shorting by contact with the support layer 66. In some embodiments, the layer of insulation 77 begins proximal of the electrode 50’ . In other embodiments, the layer of insulation 77 extends all the way through the electrode 50’ but is stripped off at a location corresponding to an aperture 72 or is removed from an outer surface of the electrode 50’ but left on an inner surface of the electrode 50’.

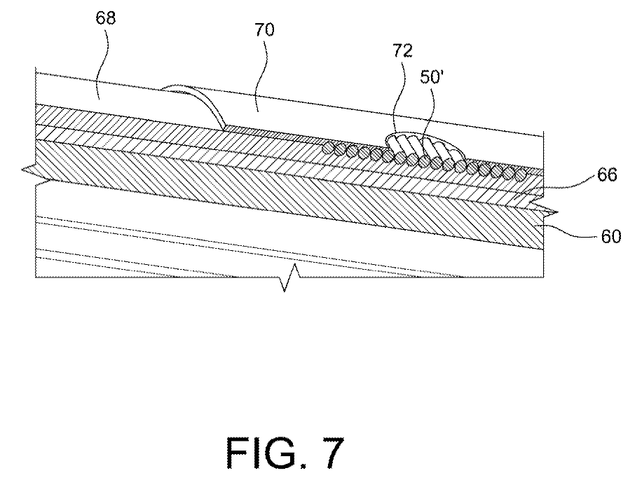

FIG. 7 is a longitudinal cross-sectional view of the catheter 32 of FIG. 6A along line 7-7, in accordance with embodiments of the disclosure. As illustrated in FIG. 7, only a portion of the electrode 50’ is exposed by the aperture 72.

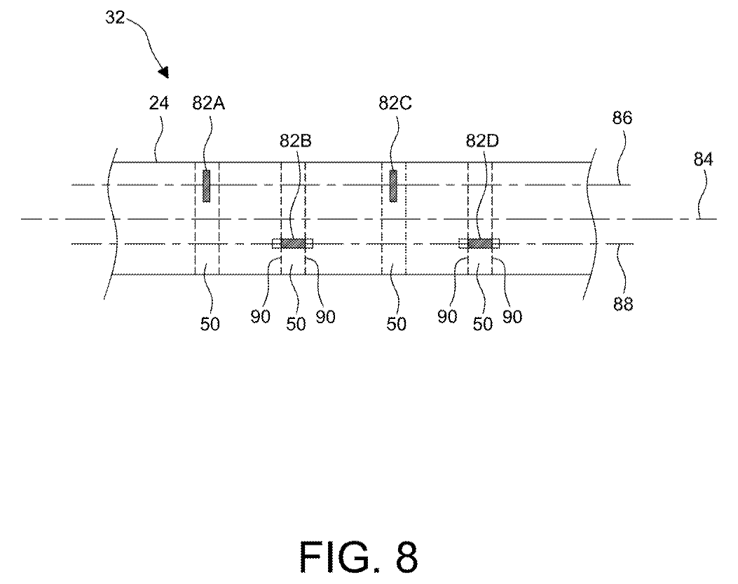

FIG. 8 is a side view of the catheter 32 illustrating an arrangement of apertures 82A, 82B, 82C, 82D exposing portions of embedded electrodes 50, in accordance with embodiments of the disclosure. As discussed previously, the apertures 82A, 82B, 82C, 82D are formed by removing a portion of the outer polymeric sleeve 70 overlaying the electrodes 50. In the embodiment of FIG. 8, four electrodes 50 take the form of solid or coiled wire ring electrodes. Each of the four electrodes 50 is exposed by a single aperture. Each aperture 82A, 82B, 82C, 82D takes the form of a rectangular slot or slit.

Apertures 82A and 82C are arranged to extend partially around a circumference of the outer member 24 and are aligned transverse to a central longitudinal axis 84 of the outer member 24. Apertures 82A and 82C share a common longitudinal axis 86 along the outer member 24 of the catheter 32. Apertures 82A and 82C have a width that is less than a width of the underlying electrode 50. In some embodiments, apertures 82A and 82C have a width that is less than a width of the underlying electrode 50 but have a length that completely circumscribes the underlying electrode exposing an entire circumferential portion of the underlying electrode 50.

Apertures 82B and 82D extend along the longitudinal axis 84 of the outer member 24 and are aligned parallel to the longitudinal axis 84. Apertures 82B and 82D share a common longitudinal axis 88 along the outer member 24 of catheter 32. Apertures 82B and 82D are larger than the portion of electrode 50 underneath. As illustrated in FIG. 8, apertures 82B and 82D extend beyond edges 90 of the underlying electrodes 50. As such, the edges 90 of the underlying electrodes 50 are exposed by the apertures 82B, 82D.

Apertures 82A and 82C are arranged at different circumferential location along the outer member 24 than apertures 82B and 82D. The common longitudinal axis 86 shared by apertures 82A and 82C is at a different circumferential location than the common axis 88 shared by apertures 82B and 82D.

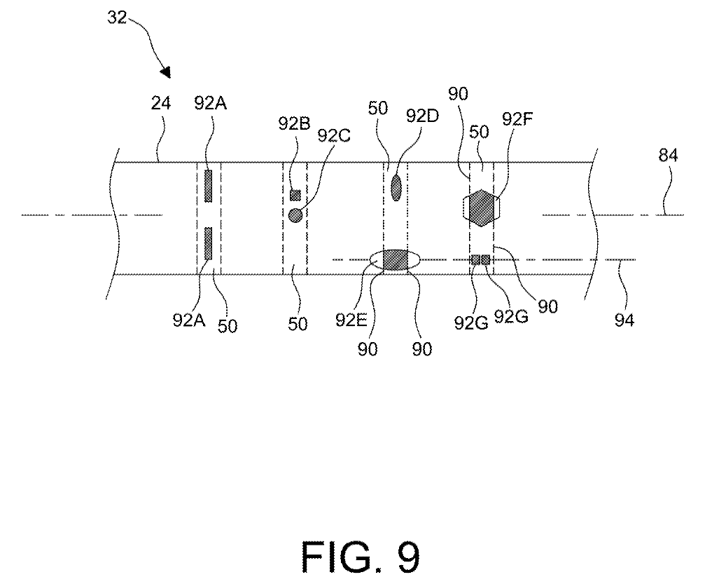

FIG. 9 is a side view of the catheter 32 illustrating an arrangement of apertures 92A, 92B, 92C, 92D, 92E, 92F, 92G exposing portions of embedded electrodes 50, in accordance with embodiments of the disclosure. As discussed previously, the apertures 92A, 92B, 92C, 92D, 92E, 92F, 92G are formed by removing a portion of the outer polymeric sleeve 70 overlaying the electrodes 50. In the embodiment of FIG. 9, four electrodes 50 take the form of solid or coiled wire ring electrodes or partial electrodes. In FIG. 9, the apertures 92A, 92B, 92C, 92D, 92E, 92F, 92G have a variety of shapes. The apertures 92A, 92B, 92C, 92D, 92E, 92F, 92G are formed as one or more slit, circle, oval, square, rectangle, or polygon.

As illustrated in FIG. 9, apertures 92A take the form of a rectangular slot or slit. The apertures 92A are both arranged over an electrode 50 at different circumferential locations exposing the electrode 50 at a plurality of circumferential locations. Apertures 92B and 92C are arranged over the same electrode 50. Aperture 92B is arranged as a square aperture exposing a first portion of the underlying electrode 50 at a first circumferential location. Aperture 92C is arranged as a circle exposing a different portion of the underlying electrode 50 at a second circumferential location. Apertures 92D and 92E are arranged over the same electrode 50. Apertures 92D and 92D are arranged as oval apertures exposing different portions of the same electrode 50. Aperture 92D is arranged transverse to a central longitudinal axis 84 of the outer member 24 while aperture 92E is arranged parallel to the longitudinal axis 84. Aperture 92E is lager than the underlying electrode 50 such that edges 90 of the electrode 50 are exposed by the aperture 92E. Aperture 92F is arranged as a polygon exposing a portion of the underlying electrode 50 as well as edges 90 of the electrode 50. Apertures 92G are arranged as square apertures sharing a common longitudinal axis with aperture 92E. As discussed previously, the common longitudinal axis can be arranged at various circumferential locations of the outer member 24, for example along a neutral axis or an axis opposite of a bend direction of the catheter 32. The apertures are arranged such that no aperture closes from compression of materials forming the outer member 24 during deflection while steering.

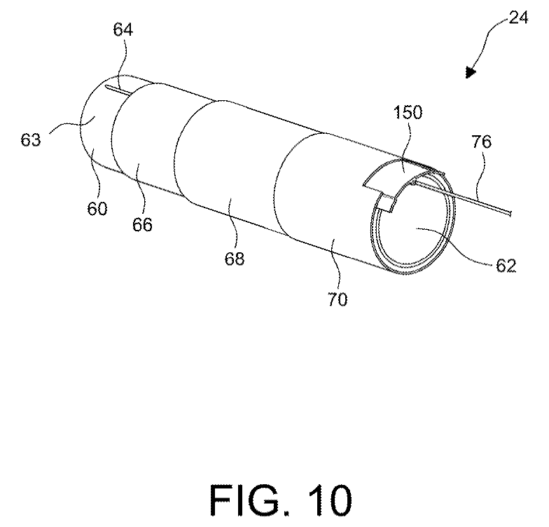

FIG. 10 is a perspective view of a portion of the outer member 24 of the catheter 32 in the intermediate region 54 illustrating at least one electrode 150 as a partial electrode. The outer member 24 is formed by a plurality of layers. The outer member 24 includes a liner 60 defining a lumen 62. The liner 60 is a polymeric liner and is formed of a suitable polymeric material such as Nylon, PEBAX, PTFE, HDPE, UHMWPE, LDPE, and MDPE. At least one guide member 64 is located along an outer surface 63 of the liner 60.

A support layer 66 surrounds the at least one guide member 64 and the liner 60. The support layer 66 includes a braided or coiled structure that provides support to the outer member 24. The support layer 66 is formed of one or more filaments or wires of a suitable polymer, metal, or fabric. The support layer 66 is formed of one or more filaments or wires of a suitable polymer, metal, or fabric. Suitable materials include stainless steel, NiTi, Nylon, Kevlar, LCP, PEEK, HDPE. In other embodiments, the support layer 66 can include a hypotube, a slotted hypotube, or a tubular member having at least one molded or laser cut articulating joint. In some embodiments, the support layer 66 entirely surrounds the at least one guide member 64. In other embodiments, the support layer 66 partially surrounds the at least one guide member 64. For example, some filaments forming the support layer 66 may go above the at least one guide member 64 while some filaments forming the support layer 66 go below the at least one guide member 64. The support layer 66 includes a material that can improve torque transmission, increase rigidity, or otherwise improve handling of the outer member 24. In some embodiments, the support layer 66 includes a consistent braid density or uniform coil pitch. In embodiments, the support layer 66 has a variable braid density or variable coil pitch. In some embodiments, the support layer 66 is formed of a slotted hypotube or tubular structure with a plurality of slots or perforations.

The at least one guide member 64 is an elongated tubular member arranged substantially parallel to a longitudinal axis of the outer member 24. The at least one guide member 64 can have a circular, rectangular, oval, square, or polygonal cross-section. The at least one guide member 64 is formed at least partially of a polymeric material, for example Pebax, PTFE, or a composite thereof. In some embodiments, the at least one guide member 64 is configured to guide at least one conductor 76 from the handle 10 to at least one electrode 150. The at least one conductor 76 electrically couples the at least one electrode 150 to a control system, for example an EAM system or an RF generator.

In other embodiments, the at least one guide member 64 is configured to guide at least one steering wire from the handle 10 to the distal end 40 of the outer member 24. The at least guide member 64 is configured to provide a channel for at least one steering wire that allows for deflection or changing shape of the outer member 24. The at least one steering wire is secured near the distal end 40 and extends proximally through the at least one guide member 64 to the handle 10. A user manipulates a control mechanism (not shown) to cause tensioning of at least one steering wire to deflect or change the shape of the outer member 24.

While not illustrated, the at least one guide member 64 can include a plurality of guide members spaced around the liner 60. In one embodiment, the plurality of guide members are equally spaced around the liner 60. In other embodiments, the plurality of guide members are adjacent one another. In some embodiments, the at least one guide member 64 includes a plurality of guide members, some of which guide a plurality of conductors, and others which guide a plurality of steering wires.

An inner polymeric sleeve 68 surrounds the liner 60, the at least one guide member 64, and the support layer 66. The inner polymeric sleeve 68 electrically insulates the support layer. The inner polymeric sleeve 68 can be applied over the liner 60, the at least one guide member 64, and the support layer 66 using a reflow process to encase the components. The inner polymeric sleeve 68 includes any polymer or polymeric material that can flow in spaces between the support layer 66 and the at least one guide member 64. Some suitable materials for forming at least a portion of the inner polymeric sleeve 68 include Nylon, PEBAX, PTFE, Polyurethane, and PET.

An outer polymeric sleeve 70 surrounds the inner polymeric sleeve 68. In some embodiments, the outer polymeric sleeve 70 is formed of a polymer having a lower melting temperature than the polymer forming the inner polymeric sleeve 68. The outer polymeric sleeve 70 provides a smooth outer surface for the outer member 24. In some embodiments, the outer polymeric layer 70 is selected to include one or more materials that increase lubricity of the catheter 32. In some embodiments, a coating (not shown) is applied over the outer polymeric sleeve 70 to increase lubricity of the medical device. In some embodiments, the outer polymeric sleeve 70 and the inner polymeric sleeve 68 are formed of the same polymer or polymeric material. In some embodiments, the outer polymeric sleeve 70 and the inner polymeric sleeve 68 are formed of different polymers or polymeric materials.

At least one electrode 150 is partially embedded in and/or mounted on the outer polymeric sleeve 70 or partially embedded in and/or mounted on the inner polymeric sleeve 68. Partially embedded in the outer polymeric sleeve 70 includes positioning partially in the sleeve 70 and positioning below the sleeve 70. Partially embedded in the inner polymeric sleeve 68 includes positioning partially in the sleeve 70 and positioning below the sleeve 68. The at least one electrode 150 is formed of an electrically conductive material. In some embodiments, the at least one electrode is formed of a metal such as a platinum-iridium alloy, gold, other platinum alloys, stainless steel, nickel-titanium or any other biocompatible medical metals. In some embodiments, the at least one electrode is formed of a conductive polymer or a polymer containing conducive particles. The at least one electrode 150 has an exposed portion spanning less than a full circumference of the outer polymeric sleeve 70 of the outer member 24. In some embodiments, an entirety of the at least one electrode 150 spans less than half of the circumference of the outer polymeric sleeve 70. In other embodiments, an entirety of the at least one electrode 150 spans less than a quarter of the circumference of the outer polymeric sleeve 70.

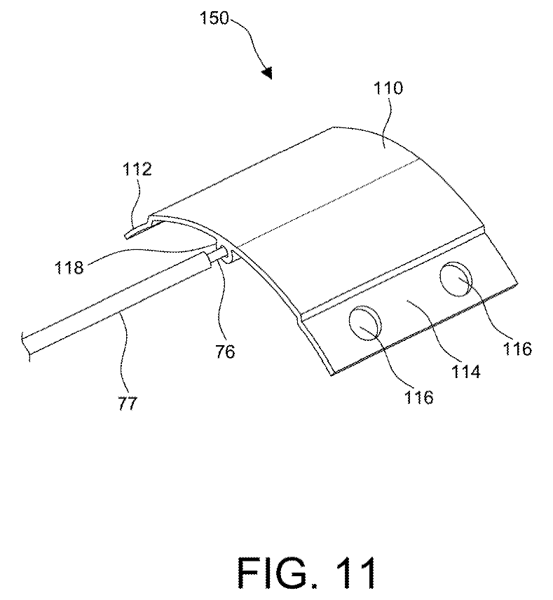

FIG. 11 is a perspective view of an electrode 150 configured as a partial electrode, in accordance with embodiments of the disclosure. The partial electrode comprises a quadrilateral and includes a main portion 110 that has a curvature corresponding to an outer circumference of the outer polymeric sleeve 70. In some embodiments, the curvature matches the curvature of the outer circumference of the outer polymeric sleeve 70. The main portion 110 includes a portion of the electrode 150 that is exposed.

The main portion 110 forms an elevated central portion of the electrode 150. The main portion 110 separates a first lower side 112 and a second lower side 114. The first lower side 112 and the second lower side 114 are recessed from or form a step down from the main portion 110. As shown in FIG. 10, the first lower side 112 and the second lower side 114 are embedded in the outer polymeric sleeve 70 while the main portion 110 extends above the sleeve 70. A plurality of openings 116 are formed through the first lower side 112 and the second lower side 114. The plurality of openings 116 are configured to enhance attachment of the electrode 150 during a reflow procedure. In some embodiments, a bottom surface 111 includes an adhesive to better adhere the electrode 150 to the outer member 24. In other embodiments, the bottom surface 111 may include an insulation layer to avoid short circuiting with metallic components of the support layer.

The electrode 150 includes a connector 118 for connecting to the at least one conductor 76. In the embodiment of FIG. 11 the connector 118 includes a central channel that receives the conductor 76. The conductor 76 is attached to the connector 118 via a crimp, adhesive, weld, friction fit, or other means for joining the conductor 76 to the channel of the connector 118.

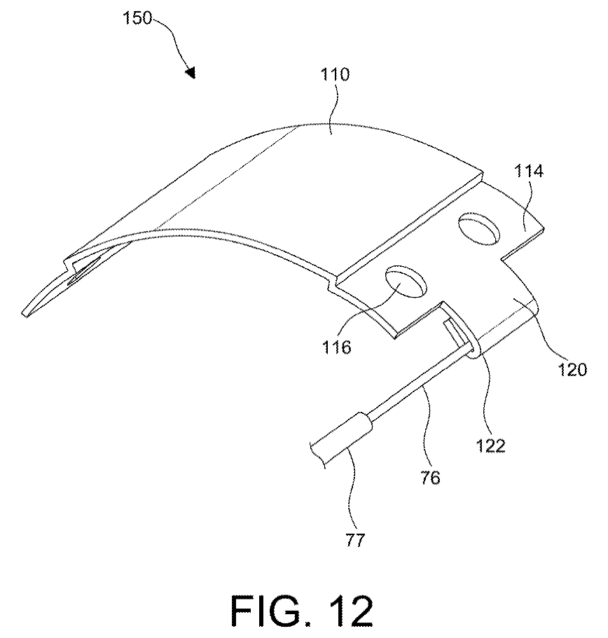

In the embodiment of FIG. 12, the connector 120 comprises a tab that is bent to retain the conductor 76. The tab is located at an edge of the first lower side 112 or the second lower side 114. The tab includes a bent portion 122 wrapping the tab around the conductor 76 and securing the conductor 76. The connector 120 allows for placement of the conductor 76 at a longitudinal axis offset from the main portion 110.

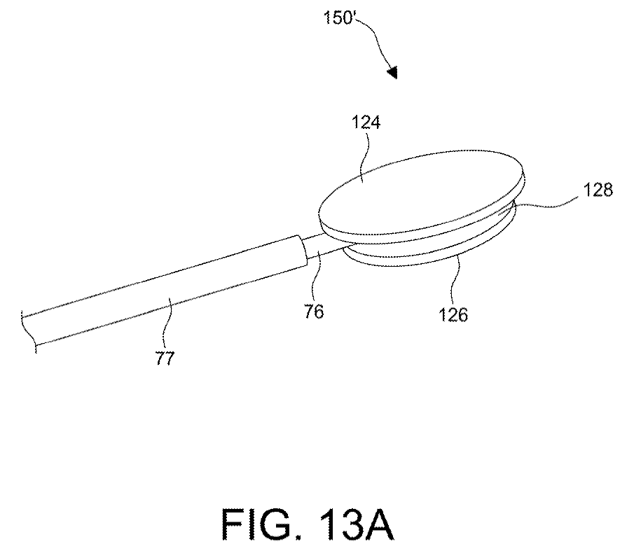

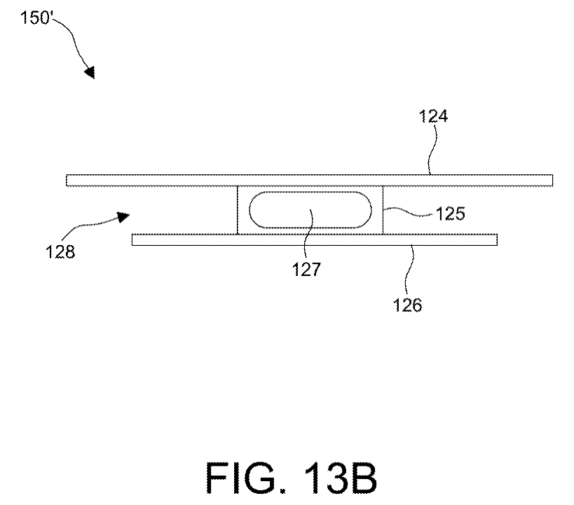

FIG. 13A is a perspective view of a partial electrode 150’, in accordance with embodiments of the disclosure. As illustrated in FIG. 13A, the electrode 150’ comprises an upper plate 124 and a lower plate 126. The lower plate 126 is slightly smaller than the upper plate 124. In some embodiments, the upper plate 124 is slightly smaller than the lower plate 126. As illustrated in FIG. 13B, the upper plate 124 is connected to the lower plate 126 by a strut 125. The strut 125 includes an opening 127 configured to receive an end of the conductor 76. During assembly, the upper plate 124 is pressed towards the lower plate 126 collapsing the strut 125 and securing the conductor 76 in the opening. A gap 128 between the upper plate 124 and the lower plate 126 is equal to or smaller than a diameter of the conductor 76. In some embodiments, the conductor 76 is flattened leading to a thinner gap. Once assembled, the upper plate 124 is exposed while the lower plate 126 is embedded in the outer polymeric sleeve 70 and the gap 128 is filled with polymer forming the outer polymeric sleeve 70. In some embodiments, the upper plate 124 and the lower plate 126 are flat. In other embodiments, the upper plate 124 and the lower plate 126 include a curvature corresponding to a curvature of the outer member 24. While the upper plate 124 and the lower plate 126 are both illustrated as circular, the upper plate 124 and the lower plate 126 can be arranged as oval, square, rectangular, polygonal, or any shape as desired. In some embodiments, the upper plate 124 and the lower plate 126 have the same size and shape. In other embodiments, the upper plate 124 has a shape or size different than the lower plate 126.

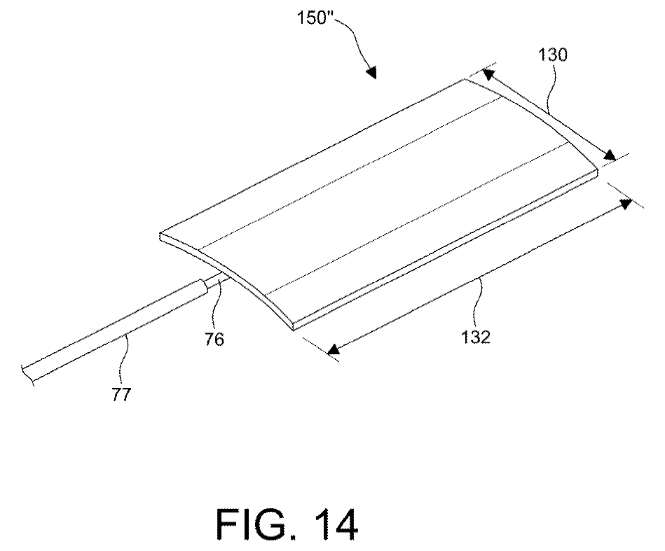

FIG. 14 is a perspective view of a partial electrode 150’’, in accordance with embodiments of the disclosure. The electrode 150’’ of FIG. 14 comprises a quadrilateral having a curve corresponding to a circumference of the outer polymeric sleeve 70. At least one conductor 76 is electrically and mechanically connected to a lower surface of the electrode 150’’. The electrode 150’’ can be attached to an outer surface of the outer polymer sleeve 70 while the conductor is embedded therein. In some embodiments, the quadrilateral has a length 132 larger than a width 130. In other embodiments, the quadrilateral has a width 130 larger than a length 132.

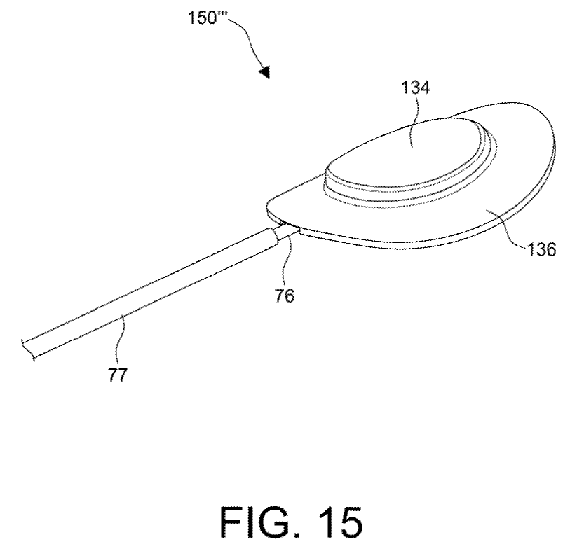

FIG. 15 is a perspective view of a partial electrode 150’’’, in accordance with embodiments of the disclosure. The electrode 150’’’ of FIG. 15 is embossed providing an elevated portion 134 and a recessed portion 136. Upon assembly, the elevated portion 134 is exposed while the recessed portion 136 is embedded in the outer polymeric sleeve 70. In the embodiment of FIG. 15, the recessed portion 136 surrounds the elevated portion 134. In other embodiments, the recessed portion 136 is surrounded by the elevated portion 134. In some embodiments, the recessed portion 136 includes a plurality of openings configured to enhance attachment of the electrode 150’’’ during a reflow procedure. In other embodiments, the electrode 150’’’ includes multiple recessed portions 136 and multiple elevated portions 134 concentrically arranged. While the electrode 150’’’ illustrated as circular, the electrode 150’’’ can be arranged as oval, square, rectangular, polygonal, or any shape as desired.

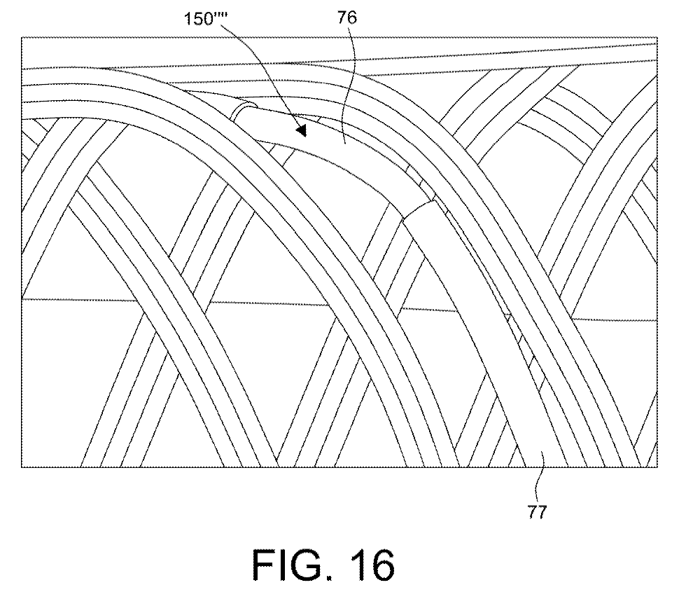

FIG. 16 is a perspective view of a partial electrode 150’’’’, in accordance with embodiments of the disclosure. In the embodiment of FIG. 16, the electrode 150’’’’ is formed by an exposed portion of the at least one conductor 76. In the embodiment of FIG. 16, a portion of a coating of insulation 77 is removed to expose the conductor 76. In other embodiments, the conductor 76 extends above the outer polymeric sleeve 70 at a desired location.

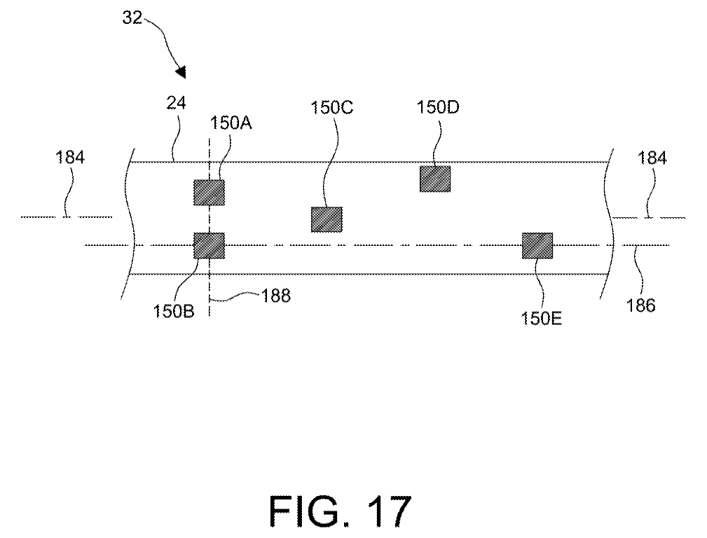

FIG. 17 is a side view of a catheter 32 illustrating an arrangement of partial electrodes 150A, 150B, 150C, 150D, 150E in accordance with embodiments of the disclosure. The partial electrodes 150A, 150B, 150C, 150D, 150E take the form of quadrilateral partial electrodes and are arranged along the outer member 24 of the catheter 32. The partial electrodes 150A, 150B, 150C, 150D, 150E are arranged at different circumferential locations along the outer member 24 of the catheter 32. Electrodes 150A and 150B share a common axis that is transverse to a longitudinal axis 184 of the catheter 32. Electrodes 150B and 150E share a common a common longitudinal axis 186 that is parallel to the longitudinal axis 184. In some embodiments, the electrodes 150A, 150B, 150C, 150D, 150E all share a common longitudinal axis that aligns with a neutral axis of the catheter 32.

While described as being incorporated into an outer member 24 of a catheter 32, the electrodes can be incorporated into any elongate medical device. For example, the electrodes described herein can be incorporated into any portion of a catheter, sheath, dilator, guide wire, stylet, obturator, lead, or any other elongate medical device.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An elongated medical device, the medical device comprising:
an inner sleeve, the inner sleeve having an outer surface extending between an inner sleeve proximal end and an inner sleeve distal end;
at least one electrode located on the inner sleeve;
at least one conductor electrically connected with the at least one electrode, the at least one conductor configured to transfer electrical signals between the at least one electrode and a control system;
an outer sleeve surrounding the at least one electrode and the inner sleeve; and
at least one aperture formed in the outer sleeve to expose a portion of the at least one electrode.

2. The medical device of claim 1, wherein the at least one electrode comprises a solid electrode or a coil electrode.

3. The medical device of claim 1, wherein the at least one electrode surrounds an entire outer circumference of the inner sleeve.

4. The medical device of device of claim 1, wherein the at least one electrode surrounds only a portion of an outer circumference of the inner sleeve.

5. The medical device of claim 1, wherein the at least one electrode includes a plurality of perforations to enhance attachment of the at least one electrode to the elongated medical device during a reflow procedure.

6. The medical device of claim 1, wherein the at least one aperture comprises one or more of a slit, circle, oval, square, rectangle, or polygon.

7. The medical device of claim 6, wherein the one or more slit extends along a longitudinal axis of the elongated medical device, or extends partially around a circumference of the elongated medical device.

8. The medical device of claim 1, wherein the at least one aperture exposes at least one edge of the at least one electrode.

9. The medical device of claim 1, wherein the at least one aperture comprises a plurality of apertures sharing a common longitudinal axis along the elongated medical device, wherein the common longitudinal axis is a neutral axis of the elongated medical device or is positioned opposite a bend direction of the elongated medical device.

10. The medical device of claim 1, wherein the at least one aperture comprises a plurality of apertures arranged at different circumferential locations along the elongated medical device.

11. An elongated medical device, the medical device comprising:
an inner sleeve, the inner sleeve extending between an inner sleeve proximal end and an inner sleeve distal end;
an outer sleeve surrounding the inner sleeve;
at least one electrode mounted on the outer sleeve, the at least one electrode having an exposed portion spanning less than a full circumference of the outer sleeve; and
at least one conductor electrically connected with the at least one electrode, the at least one conductor configured to transfer electrical signals between the at least one electrode and a control system.

12. The medical device of claim 11, wherein an entirety of the at least one electrode spans less than half of the circumference of the outer sleeve.

13. The medical device of claim 11, wherein an entirety of the at least one electrode spans less than a quarter of the circumference of the outer sleeve.

14. The medical device of claim 11, wherein the at least one electrode includes a connector for connecting to the at least one conductor, the connector comprising a bent portion at an edge of the at least one electrode or a central channel configured to receive the at least one conductor.

15. The medical device of claim 11, wherein the at least one electrode comprises a quadrilateral having a curve corresponding to a circumference of the outer sleeve, the at least one electrode comprising an elevated central portion forming the exposed portion and separating a first lower side from a second lower side, and a plurality of openings formed through at least one of the first lower side or the second lower side configured to enhance attachment of the at least one electrode during a reflow procedure.

16. The medical device of claim 11, wherein the at least one electrode comprises an upper plate forming the exposed portion and a lower plate at least partially embedded in the outer sleeve, the upper plate connected to the lower plate by a strut.

17. The medical device of claim 11, wherein the at least one electrode is embossed, the at least one electrode comprising an elevated portion forming the exposed portion and a recessed portion embedded in the outer sleeve.

18. An elongated medical device, the medical device comprising:
a liner defining a lumen, the liner extending between a liner proximal end and a liner distal end;
at least one guide member located along an outer surface of the liner;
a support layer surrounding the at least one guide member and the liner;
an inner polymeric sleeve surrounding the liner, the at least one guide member, and the support layer;
an outer polymeric sleeve surrounding the inner polymeric sleeve;
at least one electrode having an exposed portion spanning less than a full circumference of the elongated medical device;
at least one conductor located within the at least one guide member and electrically connected with the at least one electrode, the at least one conductor configured to transfer electrical signals between the at least one electrode and a control system;
an inner member movable within the lumen; and
an expandable assembly including a distally located central hub portion affixed to the inner member and a plurality of splines.

19. The medical device of claim 18, wherein the at least one electrode is located on the inner polymeric sleeve, and wherein the outer polymeric sleeve includes at least one aperture formed therein to expose the exposed portion of the at least one electrode.

20. The medical device of claim 18, wherein the at least one electrode is at least partially embedded in the outer polymeric sleeve and has an exposed portion spanning less than a full circumference of the outer polymeric sleeve.
